(19) United States
(12) Patent Application Publication
(10) Pub. No.: US 2005/0281386 A1
(43) Pub. Date: Dec. 22, 2005

(54) Dental x-ray film holder

(76) Inventor: Emad El Haje, Washington, DC (US)

Correspondence Address: Barnes & Thornburg, Suite 900, 750 17th Street, NW, Washington, DC, 20006, US

(21) Appl. No.: 10/872,521

(22) Filed: Jun. 22, 2004

(51) Int. Cl. G03C 5/16; A61B 6/14; G03B 42/02

(52) U.S. Cl. 378/168

(57) ABSTRACT

A pliant tip is provided for a dental x-ray film holder to keep the vertical plate portion of the x-ray film holder from causing discomfort to the upper roof or lower floor of a patient's mouth when the x-ray film holder with film is placed behind a patient's tooth.

### BACKGROUND AND SUMMARY OF THE INVENTION

The invention relates to a dental x-ray film holder wherein a pliant tip is provided to provide a cushioned surface for the x-ray film holder adjacent the upper or lower surfaces of a patient's mouth.

Dental x-ray film holders have a flat plate vertically extending from an end of a base handle portion. The base handle portion has a reduced thickness portion which defines a channel adjacent the vertically extending flat plate. The x-ray film is frictionally placed in the channel to rest against the vertical flat plate. The vertical flat plate is usually made from a stiff plastic material and its upper edge surface can abrade or cause discomfort to the roof of a patient's mouth when the x-ray film is placed in the patient's mouth behind the patient's teeth.

The instant invention provides for a pliant tip for the upper edge surface of the vertical plate portion of the dental x-ray film holder to reduce abrasion and discomfort to the patients upper and bottom mouth surfaces.

Other objects, advantages and novel features of the present invention will become apparent from the following detailed description of the invention when considered in conjunction with the accompanying drawings.

### BRIEF DESCRIPTION OF THE DRAWINGS

FIG. 1 shows a schematic front view of a patient's mouth;

FIG. 2 shows a side view of the patient's mouth taken along line 2-2 of FIG. 1 and with a dental x-ray film holder about to be located behind the upper teeth;

FIG. 3 shows a dental x-ray film holder with an inserted film shown in dotted lines and with a pliant tip covering about to be inserted onto the vertical flat plate portion of the x-ray film holder;

FIG. 4 shows the pliant tip portion integral with the dental x-ray film holder; and

FIGS. 5 and 6 shows two configurations of pliant tips to be slid over the flat plate portion of the dental x-ray film holder.

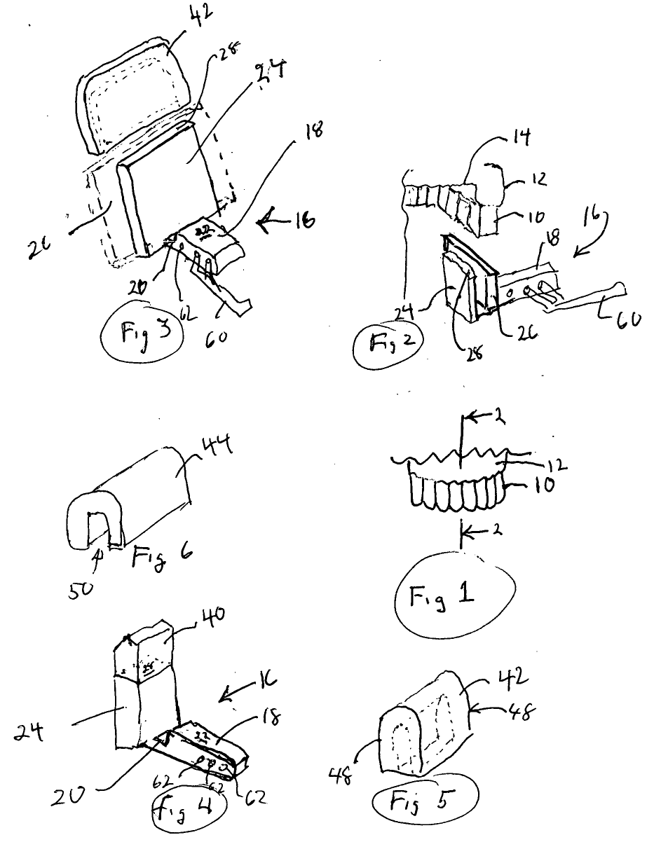

### DETAILED DESCRIPTION OF THE DRAWINGS

Looking at FIG. 2 one can see a patient's upper teeth 10 extending downward from the patient's upper gum 12 and the upper roof surface 14 of the patient's mouth.

A dental x-ray film holder 16 is shown in FIGS. 3 and 4, which holder 16 has a base portion 18 with a reduced thickness portion 20 adjacent a non reduced portion 22 and a vertical plate portion 24 adjacent the reduced thickness portion 20 such that the reduced thickness portion 20 defines a channel for reception of a dental x-ray film 26 which is frictionally held in the channel. A top edge 28 of the vertical plate portion 24 of the x-ray film holder along with the dental film 26 are inserted into a patient's mouth behind the teeth 20. A handle 60 is inserted into holes 62 of the base 18 to allow the dentist to manipulate the holder 16. Upon insertion of the holder 16 into the patient's mouth, the top edge 28 of the vertical plate portion 24 can dig into, abrade or generally cause discomfort to the roof 14 of a patient's mouth due to the rigidness of the plate portion 24.

Similarly the bottom floor of a patient's mouth could be contacted when bottom teeth x-rays are undertaken.

To avoid such discomfort, the top edge portion 28 of the vertical plate portion 24 should be pliant, so as to provide a compressible flexible soft contact with the roof 14 of the patient's mouth.

FIG. 4 shows a first embodiment wherein a pliant tip 40 is provided on the upper top edge portion 28 of the vertical plate portion 24. The tip can be attached by fusing, gluing or any similar method such that it forms an integral portion of the x-ray film holder. The tip could be made from a soft flexible or foam rubber, or plastic material, such that it could be sterilized in a dentist's autoclave.

FIGS. 5 and 6 show embodiments when the pliant material is in the form of a hood 42, 44 which slides over the upper top edge 28 of the vertical plate portion 24. FIG. 5 shows the hood to have slides 48 with a hollow center much like a pillow case, while FIG. 6 shows the hood 44 to have a slot 50 to cover the upper top edge 28 of the vertical plate portion 24. Where reusability is of no concern, the hood could be made from cotton or some other pliant inexpensive disposable material. The embodiment of FIG. 6 could be a long coiled strip of cotton where the dentist cuts off a short piece for use.

Although the present invention has been described and illustrated in detail, it is to be clearly understood that the same is by way of illustration and example only, and is not to be taken by way of limitation. The spirit and scope of the present invention are to be limited only by the terms of the appended claims.

1. A dental x-ray film holder comprising:
a base having a reduced thickness portion adjacent a non-reduced thickness portion and a vertical plate portion adjacent the reduced thickness portion of the base to cause the reduced thickness portion to define a trough between the vertical plate portion and the non-reduced thickness portion for reception of a dental x-ray film in the trough frictionally held between the vertical plate portion and the non-reduced portion thickness, and
a pliant tip on an end portion of the vertical plate portion to provide a cushion between the dental x-ray holder and a patient's mouth.

2. The dental x-ray film holder of claim 1 wherein the pliant tip is made of a material that can be autoclaved for sterility purposes without loss of pliancy.

3. The dental x-ray film holder of claim 1 wherein the plaint tip is a cover that can be slipped over the end portion of the vertical plate portion.

4. The dental x-ray film holder of claim 2 wherein the plaint tip is a cover that can be slipped over the end portion of the vertical plate portion.

5. The dental x-ray film holder of claim 1 wherein the pliant tip has a pocket which slips over the end portion of the vertical plate portion.

6. The dental x-ray film holder of claim 2 wherein the pliant tip has a pocket which slips over the end portion of the vertical plate portion.

7. The dental x-ray film holder of claim 3 wherein the pliant tip has a pocket which slips over the end portion of the vertical plate portion.

8. The dental x-ray film holder of claim 4 wherein the pliant tip has a pocket which slips over the end portion of the vertical plate portion.

9. The dental x-ray film holder of claim 1 wherein the pliant tip has an open trench therein which trench slips over the end portion of the vertical plate portion.

10. The dental x-ray film holder of claim 2 wherein the pliant tip has an open trench therein which trench slips over the end portion of the vertical plate portion.

11. The dental x-ray film holder of claim 3 wherein the pliant tip has an open trench therein which trench slips over the end portion of the vertical plate portion.

12. The dental x-ray film holder of claim 4 wherein the pliant tip has an open trench therein which trench slips over the end portion of the vertical plate portion.

13. The dental x-ray film holder of claim 1 wherein the pliant tip is made from cotton.

14. The dental x-ray film holder of claim 3 wherein the pliant tip is made from cotton.

15. The dental x-ray film holder of claim 5 wherein the pliant tip is made from cotton.

16. The dental x-ray film holder of claim 7 wherein the pliant tip is made from cotton.

17. The dental x-ray film holder of claim 9 wherein the pliant tip is made from cotton.

18. The dental x-ray film holder of claim 11 wherein the pliant tip is made from cotton.

19. The dental x-ray film holder of claim 1 wherein the pliant tip is made from plastic.

20. The dental x-ray film holder of claim 1 wherein the pliant tip is made from a formed material.

21. The dental x-ray film holder of claim 1 wherein the pliant tip is in the form of a long strip of material which is cut to form multiple individual tips.

22. The dental x-ray film holder of claim 13 wherein the pliant tip is in the form of a long strip of material which is cut to form multiple individual tips.
